(19) United States
(12) Patent Application Publication
(10) Pub. No.: US 2024/0197940 A1
(43) Pub. Date: Jun. 20, 2024

(54) SYSTEM FOR THE AUTOMATED SANITIZATION OF CONTAINERS FOR BIOLOGICAL SAMPLES AND ITS PROCESS OF USE

(71) Applicant: Robosan S.r.l., Napoli (IT)

(72) Inventors: Vittorio Trifari, Napoli (IT); Salvatore Corcione, Portici (NA) (IT); Vincenzo Cusati, Volla (NA) (IT); Danilo Ciliberti, San Paolo Del Sito (NA) (IT); Manuela Ruocco, Quarto (NA) (IT); Stanislao Grazioso, Castellamare di Stabia (NA) (IT); Daniela Terracciano, Napoli (IT); Teodorico Caporaso, Benevento (IT); Dario Panariello, Colli del Tronto (AP) (IT); Giuseppe Di Gironimo, Napoli (IT)

(73) Assignee: Robosan S.r.l., Napoli (IT)

(21) Appl. No.: 18/286,910

(22) PCT Filed: Aug. 4, 2021
(86) PCT No.: PCT/EP2021/071715

§ 371 Date: Oct. 13, 2023

(30) Foreign Application Priority Data
Apr. 13, 2021 (IT) 102021000009296

(51) Int. Cl. A61L 2/24; A61L 2/10; A61L 2/22

(52) U.S. Cl. CPC A61L 2/24; A61L 2/10; A61L 2/22; A61L 2202/14; A61L 2202/15; A61L 2202/23

(57) ABSTRACT

A system for the automated sanitization of containers for biological samples is described, the system comprising housing means for containers for biological samples, a sanitization route having an inlet opening and an outlet opening, handling means of said housing means, which are suitable for conveying the containers for biological samples along said sanitization route, a sanitization chamber and means for drying containers for biological samples; a process employing the system of the above-mentioned type is also described.

### FIELD OF APPLICATION

The present invention refers to a system, comprising mechanical and electrical components, suitable for the automated sanitization of containers for biological samples, for example closed test tubes in which a biological sample has been previously introduced.

In particular, said system comprises means for dispensing a biocidal fluid, such as for example an aqueous solution of sodium hypochlorite.

Moreover, the present invention refers to a process for the sanitization of containers for biological samples using said system.

### PRIOR ART

There is a very topical need for providing a method for collecting, handling and successively sending biological samples to an analytical laboratory, wherein said method should not endanger the health of the involved health-care professionals and, at the same time, should minimize the risk of contamination and alteration of the biological sample collected and sent to an analytical laboratory.

Specifically, in the context of the pandemic caused by the new Coronavirus Sars-COV-2, hereinafter referred to as Covid-19, there is an urgent need for ensuring handling biological samples, such as nasal, salivary or nasal-oropharyngeal swabs, capillary blood samples for rapid serological test or blood sampling, in a way which is as much free of risks for the involved health-care professionals' health as possible.

In particular, it is currently common to collect biological samples, usually nasal, salivary or nasal-oropharyngeal swabs, on-site, i.e. outside facilities which are per se designated to the subsequent step of biological analysis, and therefore it is normally necessary to provide disinfection and sanitization of the outer surface of casings or containers inside which the biological samples are placed, before sending them to an analytical laboratory or upon their arrival there.

It is indeed known that several pathogenic agents can resist on a variety of surfaces for a time period varying from hours to days. Human coronaviruses, for example, can resist on surfaces up to 9 days at room temperature.

See for example the following publications: Van Doremalen N., Bushmaker T, Morris D. H., et al., “Aerosol and surface stability of SARS-CoV-2 as compared with SARS-COV-1”, New. Eng. J. Med., 2020 March 17, 382(16): 1564-1567 (doi: 10.1056/NEJMc2004973) and Kampf G. et Al., “Persistence of coronaviruses on inanimate surfaces and its inactivation with biocidal agents”, J. Hosp. Infect., 2020 March, 104(3): 246-251.

Due to the persistence of these pathogens on surfaces there is a high biological risk in workplaces and, in particular, operators of analytical laboratories may become infected by touching contaminated surfaces.

In the absence of suitable devices, the sanitization operation is often performed by simply washing the containers containing biological samples with disinfectant solutions and/or soap by hand.

Said operation is typically performed immediately before the analysis step in the laboratory, without ensuring an upstream sanitization of the sample when it is collected, thus causing thereby the risk of a possible propagation of contamination during intermediate steps and handling between sample-taking and manual washing thereby performed.

However, manually-performed operations of sanitization of containers containing biological samples cause several setbacks.

First of all, the risk of contamination and possible infection for the health-care professional remains significant; moreover, some ways to sanitize and disinfect, which involve for example the use of a large amount of washing or disinfection fluids, as well as the use of unsuitable washing liquids such as solutions of ethyl alcohol or of hydrogen peroxide, may bring about detachment or discoloration of the label affixed on the biological sample container, thereby causing consequent loss of personal information of the patient or causing the mismatch of the biological sample and the patient's identification.

Lastly, not only is a high efficiency for operations of manual sanitization of biological samples not ensured, but this might also not be performed in an effective manner due to situations of particular urgency or negligence by the health-care professional designated for this specific operation.

Moreover, any operation of manual sanitization of containers containing biological samples brings about a significant slowing down in the operations of processing the biological sample: on the other hand, this situation is a serious disadvantage in critical situations such as the pandemic, during which processing and analysis are required to be very fast.

In the art, there are systems for washing and/or automated disinfection of test tubes or similar containers.

CN110319672 A discloses a device for washing, sanitizing and subsequently automatically drying test tubes which allows to sterilize empty test tubes using UV lamps. Drying occurs instead by hot air.

CN203803537U discloses a device to organize and hygienize empty laboratory test tubes. The test tubes are placed on a designated rack, from which they are conveyed towards a washing zone; the test tubes are then dried using an infrared lamp and finally sterilized using UV radiation.

The above-mentioned devices are thus suitable for washing, sanitizing and drying empty test tubes, but are not suitable for performing said operations on the outer surface of casings or containers containing biological samples.

In fact, whether they are made of plastic or glass, said containers for biological samples are usually not able to filter ultraviolet radiation, which would cause alteration of the biological sample placed inside them or would anyway destroy the genetic material.

In conclusion, in the art exists still an urgent need to provide a system for automated sanitization of containers containing biological samples, which is suitable for the objective and overcomes the drawbacks of the solutions previously reported with reference to the above-described prior art, in particular which solves the problems related to automation of sanitization techniques with concurrent preservation of the intactness of the biological sample.

The technical problem underlying the present invention is thus providing a system for the automated sanitization of containers containing biological samples which allows to minimize the risk of contamination for the health-care professional, thereby also ensuring a proper storage of the biological sample therein contained.

### SUMMARY OF THE INVENTION

According to the present invention, said problem is solved by a system for the automated sanitization of containers for biological samples, the system comprising housing means for containers for biological samples, a sanitization route having an inlet opening and an outlet opening, handling means of said housing means, which are suitable for conveying the containers for biological samples along said sanitization route, a sanitization chamber comprising means for dispensing a biocidal fluid, and means for drying containers for biological samples.

According to the present invention, the expression “containers for biological samples” means containers suitable for storage and transportation of biological samples, such as nasal, salivary or nasal-oropharyngeal swabs, for example swabs for Covid-19 diagnosis, capillary blood samples for rapid serological test, blood sampling or urine samples, made of plastic material or glass, for example in the form of a test tube, which typically comprise an elongated vessel having two extremities, a first extremity which is closed, placed lower than the other and optionally tapered, suitable for housing it inside a secondary container for the transportation of containers for biological samples, and a second extremity which is open, placed higher than the first, but which can be closed by a sealing cap, for example provided with a threaded rim on which the cap can be screwed.

According to the present invention, the containers for biological samples are typically containers containing biological sample, i.e. in which the vessel contains inside it a sample of biological liquid and/or tissue, possibly suitably diluted and/or stored in a physiological solution and/or a cell culture medium, the vessel being sealed by the cap.

According to the present invention, the biocidal fluid is an aqueous solution of sodium hypochlorite, in particular an aqueous solution having a sodium hypochlorite concentration between 0.2% and 0.6%, more in particular of 0.5% by weight of the total weight of the solution.

According to the present invention, the above-mentioned handling means of said housing means for conveying the containers for biological samples along said sanitization route are suitable for handling these housing means and, optionally, are means for linear handling.

Advantageously, as it will also be clear below with reference to some preferred embodiments, the system according to the present invention allows to sanitize in an automated manner the above-mentioned containers for biological samples, thereby minimizing the risk of contamination for the health-care professional, who will have to perform as few operations of direct handling of the non-sanitized container for biological samples as possible, and at the same time preserving the intactness of the biological sample therein contained.

Accordingly, the system according to the present invention allows to sanitize in an automated manner the above-mentioned containers for biological samples by killing almost completely all viruses, bacteria, fungi and/or other micro-organisms eventually present on the external surface of these containers for biological samples.

In particular, the system according to the present invention allows to sanitize in an automated manner the above-mentioned containers for biological samples by killing viruses, more in particular Coronavirus Sars-COV-2, eventually present on the external surface of these containers for biological samples.

Preferably, the sanitization chamber may comprise said means for drying containers for biological samples.

More preferably, said means for dispensing a biocidal fluid are placed upstream of said means for drying containers for biological samples along the above-mentioned sanitization route.

Alternatively, the above-mentioned system for the automated sanitization of containers for biological samples may preferably comprise a drying chamber wherein said means for drying containers of biological samples are placed.

Precisely, when the system according to the present invention comprises a drying chamber, the sanitization chamber is placed upstream of the same along said sanitization route.

Generally speaking, the sanitization chamber and the drying chamber, when present, are in fact placed along said sanitization route.

Preferably, the above-mentioned means for dispensing a biocidal fluid are a plurality of atomizer nozzles.

More preferably, the atomizer nozzles comprise a biocide-fluid-dispensing hole for a jet of biocidal fluid.

Even more preferably, said dispensing hole for a jet of biocidal fluid has a shape suitable for providing the dispensation of a blade-shaped jet of biocidal fluid.

Preferably, said dispensing hole for a jet of biocidal fluid is suitable for dispensing a jet of biocidal fluid having an average size of fluid particles between 25 and 45 microns, more preferably of 30 microns.

Advantageously, the provision of a jet of biocidal fluid with an average size of fluid particles equal to the above-specified values allows an appropriate adhesion of the biocidal fluid particles to the walls of the containers for biological samples to be sanitized.

Even more preferably, said sanitization chamber comprises two walls opposite each other on which said atomizer nozzles are located.

Most preferably, the atomizer nozzles are at least two, each of which being respectively placed on one of said two walls opposite each other of the sanitization chamber.

Preferably, according to this last embodiment, when said housing means are in the sanitization chamber, said at least two nozzles are suitable for dispensing a blade-shaped jets of biocidal fluid.

Advantageously, in this way the at least two nozzles cause the impact between said blade-shaped jets of biocidal fluid and opposite surfaces of said containers for biological samples.

More preferably, the above-mentioned atomizer nozzles allow the dispensation of jets of biocidal fluid having a jet opening angle between 60° and 90°, even more preferably between 65° and 85°.

Preferably, the system according to the present invention further comprises a reservoir suitable for containing said biocidal fluid.

More preferably, said reservoir contains biocidal fluid, the latter being an aqueous solution of sodium hypochlorite, in particular an aqueous solution having a concentration of sodium hypochlorite between 0.2% and 0.6%, more in particular of 0.5% by weight of the total weight of the solution.

Preferably, said means for drying containers for biological samples are suitable for dispensing a gas flow, more preferably gas at room temperature, most preferably compressed air, for example air for medical use.

More preferably, said means for drying, suitable for dispensing a gas flow, are a plurality of drying nozzles.

Even more preferably, the above-mentioned drying nozzles allow the dispensation of jets of a gas flow having a jet opening angle between 0° and 120°, most preferably of 120°.

Consistently, the system according to the present invention may comprise a reservoir for compressed air, for example air for medical use.

Preferably, said sanitization route is entirely comprised between the above-mentioned inlet opening and said outlet opening.

According to one of its preferred embodiments, the system of the present invention comprises a packaging zone comprising means for packaging said containers for biological samples.

In particular, said means for packaging are suitable for packaging, and possibly loading, containers for biological samples in a secondary container for the transportation of containers for biological samples.

Preferably, said means for packaging are suitable for sealing, optionally air-tight sealing, said secondary container for the transportation of containers for biological samples.

Advantageously, in a totally automated manner, once the above-mentioned containers for biological samples have been placed into the secondary container for the transportation of containers for biological samples, the system according to the present invention is able to seal, optionally air-tight seal, the latter, so as to prevent the contamination of said previously-sanitized containers for biological samples during subsequent transportation operations.

According to the present invention, the expression “secondary container for the transportation of containers for biological samples” means a secondary vessel which consists in an impermeable, air-tight, container made of a resistant material, suitable to contain and protect one or more primary vessels (as long as individually wrapped in absorbent material); outside said material should be applied cards with the data identifying and describing the contents, the sender and the recipient (as provided, in Italy, by the Circular n. 16 of the Ministry of Health of 1994 and by the more recent Circular n. 3 of May 8, 2003).

In particular, said secondary container for the transportation of containers for biological samples comprises a support base and a lid.

More in particular, said secondary container for the transportation of containers for biological samples may normally be air-tight sealed by a gasket and/or by suitable safety hooks made in the base and/or in the lid of the secondary container.

Inside the above-mentioned support base, said secondary container for the transportation of containers for biological samples may further comprise a grid-shaped container holder, optionally made of absorbent material, which is able to hold the containers containing biological samples in a vertical position, so as to ensure proper storage and transportation thereof.

Specifically, said grid-shaped container holder, optionally made of absorbent material, comprises designated receiving wells in which containers for biological samples, such as test tubes, may be placed.

More specifically, said grid-shaper container holder is suited to receiving containers for biological samples suitable for storage and transportation of biological samples, such as nasal, salivary or nasal-oropharyngeal swabs, for example swabs for Covid-19 diagnosis, capillary blood samples for rapid serological test, blood sampling or urine samples, made of plastic material or glass, typically test tubes.

It is to be understood that, according to the present invention, said secondary container for the transportation of containers for biological samples is not comprised in the system according to the present invention.

Preferably, the system according to the present invention comprises a sterilization chamber for said secondary container for the transportation of containers for biological samples, wherein said sterilization chamber comprises sterilization means for a secondary container for the transportation of containers for biological samples which are suitable for sterilizing the surface of said secondary container for the transportation of containers for biological samples.

More preferably, said sterilization means for a secondary container for the transportation of containers for biological samples are ultraviolet light lamps.

Even more preferably, said sterilization chamber for a secondary container for the transportation of containers for biological samples comprises at least one inlet opening.

In particular, the sterilization chamber comprises a first inlet opening, suitable to allow the operator to insert the support base of the secondary container for the transportation of containers for biological samples into the sterilization chamber, and a second inlet opening, suitable to allow the operator to insert the lid of the secondary container for the transportation of containers for biological samples into the sterilization chamber.

Preferably, the system according to the present invention comprises transportation means for a secondary container for the transportation of containers for biological samples, which are able to convey in an automated manner said secondary container for the transportation of containers for biological samples from the sterilization chamber to the packaging zone and/or from the packaging zone towards the above-mentioned outlet opening of the sanitization route for taking the sanitized containers.

More preferably, said transportation means for a secondary container for the transportation of containers for biological samples are able to convey in an automated manner the base and/or the lid of the above-mentioned secondary container for the transportation of containers for biological samples from the sterilization chamber to the packaging zone and/or from the packaging zone towards the outlet opening of the sanitization route.

According to a preferred embodiment, the system according to the present invention may comprise a separation element, placed downstream of the sanitization chamber and upstream of the packaging zone along said sanitization route, more preferably wherein said separation element acts as a barrier for said biocidal fluid.

Most preferably, said separation element may be made of plastic or metal rigid material or of flexible material, for example flexible plastic material such as PVC.

When said separation element is made of a plastic or metal rigid material, said small door may be a hatch.

Most preferably, said hatch may be a sliding hatch or a hinged hatch, for example a double leaf hinged hatch.

Preferably, said separation element is located between the above-mentioned sanitization chamber and the packaging zone; more preferably, between the above-mentioned drying chamber, if present, and the packaging zone.

Advantageously, said separation element hinders leakage of droplets of biocide liquid from the sanitization chamber, i.e., more in general, the dispersion of droplets of biocide liquid in areas of the system according to the invention which are placed downstream of the sanitization chamber along the sanitization route.

According to another preferred embodiment, the above-mentioned housing means for containers for biological samples comprise a drilled plate comprising through holes which are suitable for receiving said containers for biological samples.

Specifically, each through hole of the drilled plate is conveniently dimensioned to house a single container for biological samples.

Preferably, said through holes are suited to receiving containers for biological samples suitable for storage and transportation of biological samples, such as nasal, salivary or nasal-oropharyngeal swabs, capillary blood samples for rapid serological test, blood sampling or urine samples, made of plastic material or glass, more preferably test tubes.

According to the present invention, the above-mentioned through holes are suitable for receiving containers for biological samples in a vertical position.

Advantageously, the fact that it is possible to hold the containers for biological samples in a vertical position makes the present system very suitable for sanitization and treatment of containers for nasal, salivary or nasal-oropharyngeal swabs, for example swabs for Covid-19 diagnosis.

Preferably, said drilled plate has an oblong form, in particular it is in the form of an oblong flat geometric shape, more preferably it has a rectangular form.

In an equally preferred manner, said through holes are longitudinally aligned along the length of the drilled plate i.e. along the horizontal symmetry axis of said oblong flat geometric shape.

According to the present invention, said drilled plate may be made of metal material, such as aluminum, or plastic material, more preferably of PMMA or ABS/PLA.

In particular, said drilled plate may be obtained by 3D printing or laser cutting techniques.

Preferably, said drilled plate comprises two portions which can be separated from each other along a separation line defined by the horizontal symmetry axis of said oblong, more preferably rectangular, shape.

Alternatively, said drilled plate comprises three portions which can be separated from each other along two separation lines defined horizontally along said oblong, more preferably rectangular, shape.

In other words, according to the latter embodiment having three portions which can be separated from each other, said two separation lines are provided longitudinally along said oblong, more preferably rectangular, shape.

More preferably, the above-mentioned housing means for containers for biological samples comprise also a supporting frame, wherein the latter is suitable for supporting said two portions which can be separated of the drilled plate and inside which the two portions which can be separated can slide along an horizontal direction.

In particular, when said two portions which can be separated come closer or move apart from each other, the containers for biological samples housed therein can be held or released, respectively.

Indeed, said separation line goes through the through holes of the drilled plate so as to allow the opening of the through holes when said two portions are separated and moved apart.

Advantageously, said drilled plate comprising two portions which can be separated from each other along a separation line defined by the horizontal symmetry axis of the oblong flat geometric shape allows to firmly hold and readily and immediately release the containers for biological samples housed in the through holes of the drilled plate.

In particular, in a thus totally automated manner, the above-mentioned containers for biological samples may be released from said drilled plate directly into the above-mentioned secondary container for the transportation of containers for biological samples i.e. individually into the receiving wells of the grid-shaped container holder of its support base.

Moreover, thanks to the release ensured by said drilled plate, the containers for biological samples can be held in a vertical position also during this operation, thereby making the present system very suitable for sanitization and treatment of containers for nasal, salivary or nasal-oropharyngeal swabs, for example swabs for Covid-19 diagnosis.

In fact, once the support base of the secondary container for the transportation of containers for biological samples is placed into the above-mentioned packaging zone, by separating and moving apart from each other said at least two portions which can be separated of the drilled plate, due to gravity the containers for biological samples may slip directly into said receiving wells, and, thus, be properly placed into said secondary container for the transportation of containers for biological samples.

In other words, said housing means for containers for biological samples in the form of a drilled plate allow to automatically release and place said containers for biological samples into said secondary container for the transportation of containers for biological samples.

By said means for packaging, the system according to the present invention is thus able to subsequently seal, in particular air-tight seal, the secondary container for the transportation of container for biological samples as previously explained.

According to a preferred embodiment of the present system, the above-mentioned handling means of said housing means are a guide which can allow the above-mentioned supporting frame of the housing means to be conveyed from said inlet opening along a sanitization route.

According to one of its preferred embodiments, the system according to the present invention comprises a loading zone located close to the above-mentioned inlet opening of the sanitization route, said loading zone being suitable for allowing to temporarily house containers for biological samples before placing them in the above-mentioned housing means.

Preferably, said loading zone comprises loading means for temporarily housing containers for biological samples.

More preferably, said loading means are a loading grid comprising slits, which have a rest end and an end-stroke end, and loading elements suitable for temporarily housing containers for biological samples, wherein each loading element is conveniently dimensioned to house a single container for biological samples and can slide inside said slits from said rest end to said end-stroke end.

At the same time, said loading means may comprise belts or conveying belts suitable for handling said loading elements from said rest end to said end-stroke end and/or from said end-stroke end to said rest end.

According to a preferred embodiment, said loading elements can freely slide inside said slits, i.e., once they are loaded with a container for biological samples, said loading elements are able to passively slide from said rest end to said end-stroke end.

In fact, said loading slits may have an appropriate inclination so as to allow said loading elements, once the latter are loaded with a container for biological samples, to passively slide from said rest end to said end-stroke end due to gravity.

Moreover, according to the present invention, the loading elements are suitable for holding the containers for biological samples in vertical position.

Advantageously, the fact that it is possible to hold the containers for biological samples in a vertical position makes the present system very suitable for sanitization and treatment of containers for nasal, salivary or nasal-oropharyngeal swabs, for example swabs for Covid-19 diagnosis.

In a totally general manner, said end-stroke end is conveniently located close to the above-mentioned inlet opening. Consistently, said rest end is in a position opposite to said end-stroke end and to the above-mentioned inlet opening.

Preferably, the system according to the present invention may comprise means for transferring containers for biological samples from said loading elements, which have conveniently reached said end-stroke end, i.e. are in an end-stroke position, to the above-mentioned housing means for containers for biological samples.

Preferably, said means for transferring containers for biological samples are a gripping module, more preferably a gripper.

More preferably, said gripper may be a gripper provided with flexible gripping fingers, i.e. a soft gripper provided of soft fingers, claw gripper, a vacuum gripper or a magnetic gripper.

Even more preferably, the gripper is a gripper provided with flexible gripping fingers, most preferably a gripper provided with active flexible gripping fingers, inflatable using compressed air, or a gripper provided with passive flexible gripping fingers, whose opening and closing is driven by an electrical motor placed inside the gripper.

In particular, the above-mentioned gripping module is able to move according to three degrees of freedom in the space, i.e. according to three predetermined handling routes along three directions which are perpendicular to each other, wherein a first and a second direction allow to properly transfer and position a container for biological samples above the respective through hole of the drilled plate, whereas the third direction, which is perpendicular to a plane of handling defined by said first and second directions, allow to insert and properly house each single container for biological samples in a corresponding through hole of the drilled plate.

Advantageously, according to the latter embodiment of the system of the present invention, the operator can load the containers for biological samples one by one into the loading zone, the system being able to automatically handle each single loading element, once it is loaded with a container for biological samples, from said rest end to said end-stroke end, as well as to subsequently transfer, also automatically, from said loading element in end-stroke position to said means housing for containers for biological samples, while the operator is loading the next container for biological samples, thereby providing a considerable time saving and a complete automation in the loading zone.

Alternatively, in a further embodiment of the present system, the above-mentioned housing means comprising a drilled plate can be suitable for gripping and transferring the above-mentioned containers for biological samples.

The above-mentioned drilled plate is therefore able to pick up and transfer the containers for biological samples, for example by picking up and transferring simultaneously eight test tubes, so as to transport them through the above-mentioned inlet opening of the sanitization route and along the same.

Specifically, bringing closer and moving apart the above-mentioned two portions which can be separated of the drilled plate allows in this case also to pick up the containers for biological samples, possibly from the above-mentioned loading means, in addition to subsequently transfer and release the same into the secondary container for the transportation of biological samples.

Advantageously, the latter embodiment is very simple and convenient since it does not include means essentially designated for the single transfer of containers for biological samples for containers for biological samples, such as for example grippers.

Moreover, according to the latter embodiment, there is also no need for providing loading means comprising the above-mentioned loading slits and the above-mentioned conveying belts for handling the containers for biological samples previously described.

Alternatively, the above-mentioned loading zone is suitable for processing randomly-organized containers for biological samples.

Preferably, the loading zone comprises a storing compartment accessible to the operator, for example in the form of a drawer.

More preferably, the loading zone also comprises means for picking-up, suitable for picking up said containers for biological samples one at a time from the storing compartment and for conveying it towards the inlet opening of the sanitization route.

Even more preferably, the above-mentioned means for picking-up also comprises a lifting mechanism, suitable for taking the containers for biological samples one by one from the above-mentioned storing compartment.

Most preferably, the storing compartment comprises a base floor provided with an appropriate slope and a bottom wall, perpendicular to said base floor, wherein the above-mentioned lifting mechanism is positioned close to the zone of junction between said base floor and the above-mentioned bottom wall.

Advantageously, in fact, the slope of the base floor of the storing compartment allows to convey the containers for biological samples towards the bottom wall.

Most preferably, the above-mentioned loading zone further comprises a ramp, suitable for receiving one by one the containers for biological samples taken by the lifting mechanism, wherein the ramp comprises a first end in communication with the lifting mechanism and a second end, located close to the opening of the above-mentioned sanitization route.

In particular, the above-mentioned ramp may be a ramp able to allow to handle the containers for biological samples or test tubes by gravity, or, alternatively, it may be a system comprising two actuated belts which, being in contact with the walls of the containers for biological samples or test tubes, allow to handle the latter towards the above-mentioned second end of the ramp.

Preferably, also according to this embodiment, the present system may comprise means for transferring containers for biological samples which are suitable for transferring the containers for biological samples from the loading zone, in particular from said second end of the above-mentioned ramp, to the above-mentioned housing means.

More preferably, said means for transferring containers for biological samples are a gripping module, more preferably a gripper.

Preferably, according to the latter embodiment, the above-mentioned loading zone suitable for processing randomly-organized containers for biological samples may comprise an additional storing compartment comprising a base floor provided with an appropriate slope and a bottom wall, perpendicular to said base floor, as well as a selecting opening, positioned close to the zone of junction between said base floor and the above-mentioned bottom wall, wherein said selecting opening is conveniently dimensioned to allow the passage of a single container for biological samples at a time.

Advantageously, in fact, the slope of the base floor of the additional storing compartment allows to convey the containers for biological samples towards the bottom wall.

In particular, the above-mentioned selecting opening is in communication with the above-mentioned storing compartment.

Therefore, once arrived towards the bottom wall of the additional storing compartment, the containers for biological samples, one by one, fall into the selecting opening and can therefore be conveyed, one by one and in an orderly manner, into the above-mentioned storing compartment, in which the above-mentioned means for picking-up is placed and operative.

Advantageously, in general, the just described embodiment of the system according to the present invention, comprising a loading zone suitable for processing randomly-organized containers for biological samples, is very useful in the field of logistics between different analytical laboratories or between the places where the samples are collected and the analytical laboratories.

In particular, the latter embodiment is very useful for big analytical laboratories where both the step of collecting biological samples followed by putting them inside containers for biological samples, and the analysis of the same biological samples are performed, thereby making the packaging of the containers for biological samples inside a secondary container for the transportation of containers for biological samples not necessary.

The latter embodiment is very useful also for centers where samples are collected before sending them to the analytical laboratory.

Also alternatively, according to a different embodiment, in the system of the present invention the above-mentioned loading zone comprises a housing compartment suitable for containing a rack for containers for biological samples.

In particular, the rack for containers for biological samples allows to hold the latter in a vertical position and is conveniently filled before being placed into the above-mentioned housing compartment by the operator.

Advantageously, according to this alternative embodiment of the present system, the operator may allocate into the loading zone a high number, for example forty, of the above-mentioned containers for biological samples by a single operation, i.e. by putting the above-mentioned rack for containers for biological samples into the housing compartment; the means for transferring containers for biological samples may then transfer the containers for biological samples from the loading zone to the above-mentioned housing means.

Clearly, according to this embodiment, the above-mentioned means for transferring containers for biological samples, which are suitable for transferring the containers for biological samples from the loading zone, are suitable for transferring these containers from the above-mentioned rack for containers for biological samples to the above-mentioned housing means.

Preferably, the system according to the present invention may be provided with designated scanning means suitable for reading labels or tags present on the above-mentioned containers for biological samples.

More preferably, said scanning means are optical scanners, laser scanners or scanning systems, in particular based on images and therefore provided with designated video cameras.

Even more preferably, said scanning means are located upstream of the inlet opening of the sanitization route and/or said scanning means are located between the zone for loading containers for biological samples and the inlet opening of the sanitization route.

In particular, said scanning means are fixed, i.e. are anchored to a structural element of the system according to the present invention.

Advantageously, said scanning means enable the tracking of the containers for biological samples processed according to the present invention along said sanitization route.

More advantageously, said scanning means enable the traceability of the containers for biological samples processed according to the present invention.

Moreover, the use of said scanning means allows to acquire information of each container for biological samples and, therefore, of each biological sample contained therein, and to store it in a data storage unit of the system according to the present invention, thereby enabling to trace the same during the whole working cycle inside the system according to the present invention until the final packaging.

Said information acquired by said scanning means and stored in the data storage unit may concern, for example, the serial identification number of the biological sample, the type of biological sample, the date when it has been collected and the type of analysis to be performed.

Accordingly, the secondary container for the transportation of containers for biological samples is marked with a unique identification code to retrieve the information related to the primary containers inside it.

According to the present invention, the system according to the present invention may comprise an electrical motor, possibly a plurality of electrical motors, and specific actuators, designated for the activation of the above-mentioned components of the present system, including the housing means for containers for biological samples, the handling means of said housing means, the means for dispensing biocidal fluid and the means for drying containers for biological samples, as well as the above-mentioned packaging means, the above-mentioned transportation means for a secondary container for the transportation of containers for biological samples, the above-mentioned sterilization means for a secondary container for the transportation of containers for biological samples, the above-mentioned loading elements and means for transferring containers for biological samples, which are included in preferred embodiments of the present invention.

In particular, the system may also comprise a plurality of sensors, for example photocells, distance sensors and proximity sensors, chambers, as well as conventional automation elements as end of stroke.

According to a further preferred embodiment, the system according to the present invention comprises a central control unit, in turn comprising at least one control card able to generate a command of activation of the above-mentioned components of the present system, including housing the means for containers for biological samples, the handling means of said housing means, the means for dispensing biocidal fluid and the means for drying containers for biological samples, as well as the above-mentioned packaging means, the above-mentioned transportation means for a secondary container for the transportation of containers for biological samples, the above-mentioned sterilization means for a secondary container for the transportation of containers for biological samples, the above-mentioned loading elements and means for transferring containers for biological samples, which are included in preferred embodiments of the present invention.

According to a preferred embodiment of the system according to the invention, the central control unit may comprise a data storage unit suitable for storing an operative program of functioning of the system itself, for example a program according to which appropriate movement and halt times for the above-mentioned components of the present system are provided.

Preferably, said data storage unit is also suitable for saving and storing information of each container for biological samples and, therefore, of each biological sample therein contained, thereby acquired by said scanning means.

According to the present invention, the present system may also comprise a terminal comprising an interface for the interaction with an operator.

Advantageously, using said terminal the operator can send commands to the above-mentioned central control unit, for example commands such as to select an operative program of functioning of the system itself and/or commands of activation and direct control of the above-mentioned components of the present system.

Preferably, said interface is suitable for audio-visually displaying information, for example by a touch-screen display.

Most preferably, therefore, the system according to the present invention is particularly suitable for the sanitization of and, more in general, for processing containers for biological samples in the form of test tubes, specifically containers for nasal, salivary or nasal-oropharyngeal swabs, for example swabs for Covid-19 diagnosis, capillary blood samples for rapid serological test, blood sampling or urine samples.

However, according to the present invention, it is not excluded that the present system, as well as all the above-mentioned components therein comprised, is suitable for the sanitization of and, more in general, for processing containers for biological samples, for example urine containers, or bags, for example bags for collecting hematic liquids, such as blood and blood components.

According to the present invention, the above-mentioned technical problem is also solved by a process for the automated sanitization of containers for biological samples performed using any of the embodiments of the system of the invention.

The process of the invention comprises the following steps:
loading containers for biological samples into housing means;
conveying said so-loaded housing means along a sanitization route using handling means of said housing means and, using means for dispensing a biocidal fluid which are placed inside a sanitization chamber, applying a biocidal fluid, preferably a jet of biocidal fluid, onto said containers for biological samples;
drying said containers for biological samples, thus providing containers for biological samples thereby sanitized and dried;
optionally, transferring said containers for biological samples thereby sanitized and dried into a secondary container for the transportation of containers for biological samples.

Preferably, the biocidal fluid is an aqueous solution of sodium hypochlorite.

More preferably, the biocidal fluid is an aqueous solution having a concentration of sodium hypochlorite between 0.2% and 0.6%, more preferably of 0.5% by weight of the total weight of the solution.

Advantageously, by providing a step of sanitization by a biocidal fluid of the above-mentioned type, the process according to the present invention brings about an efficient and rapid sanitization of the outer surface of the above-mentioned containers for biological samples without altering nor damaging the biological sample therein stored.

Moreover, exactly thanks to the use of an aqueous solution of sodium hypochlorite, the present process does not cause any discoloration of possible labels or tags present on the outer surface of the above-mentioned containers for biological samples.

In addition, also thanks to the use of an aqueous solution of sodium hypochlorite, the present process does not cause phenomena of corrosion of the metal components with which the system according to the present invention is made, for example of the above-mentioned housing means, which may be made as a drilled plate made of metal.

In an equally preferred manner, in said step of applying a biocidal fluid a jet of biocidal fluid is applied and said means for dispensing a biocidal fluid are a plurality of atomizer nozzles.

More preferably, the biocidal fluid is dispensed by said atomizer nozzles as a blade-shaped jet.

Preferably, said jet of biocidal fluid has an average size of fluid particles between 25 and 45 microns, more preferably of 30 microns.

Advantageously, the provision of a jet of biocidal fluid with an average size of fluid particles equal to the above-specified values allows an appropriate adhesion of the biocidal fluid particles to the walls of the containers for biological samples to be sanitized.

According to a particular embodiment of the present process, in said step of applying a jet of biocidal fluid the biocidal fluid is dispensed by said atomizer nozzles as a jet having a jet opening angle between 60° and 90°, preferably between 65° and 85°.

According to a most preferred embodiment of the present process, in the step of applying a jet of biocidal fluid, the containers for biological samples are conveyed along said sanitization route according to a step-by-step movement of said handling means and in such a way that the containers for biological samples are entirely hit by the jet of biocidal fluid, gradually all over their height.

Advantageously, according to the latter embodiment of the present process, an almost total sanitization of the outer surface of the containers for biological samples is ensured, i.e. in the case of test tubes from their lower end to their upper end at which the cap is placed.

Preferably, after said step of applying a biocidal fluid and before the following drying step, the biocidal fluid is let act for a time period between 30 seconds and 2 minutes, more preferably for about 1 minute.

Preferably, the drying step is performed by dispensing compressed air, more preferably air for medical use.

More preferably, the drying step is performed by dispensing compressed air at room temperature.

According to the present invention, the expression “room temperature” means a temperature between 5° ° C. and 25° C., more precisely of about 20° C.

Even more preferably, the drying step is performed by dispensing compressed air by drying nozzles, which allow the dispensation of jets of a gas flow having a jet opening angle between 0° and 120°, most preferably of 120°.

Advantageously, by providing a drying step performed by dispensing compressed air at room temperature, the process according to the present invention brings about an efficient and rapid drying of the outer surface of the above-mentioned containers for biological samples without altering nor damaging the biological sample therein stored.

Even more preferably, in said step of applying a biocidal fluid, by mixing with the biocidal fluid, possibly inside the above-mentioned atomizer nozzles, and/or in said drying step, compressed air is dispensed at a pressure between 0.5 bar and 2 bar.

In particular, with reference only to the step of applying biocidal fluid, the pressure of dispensation of compressed air, mixed with the biocidal fluid, may be functional to obtain a jet of biocidal fluid with an average size of fluid particles equal to the above-mentioned values.

In an equally preferred manner, in said drying step compressed air is dispensed for a time period between 15 seconds and 2 minutes, most preferably of 30 seconds.

Moreover, according to the process described for the present invention, the above-mentioned steps of loading the housing means, conveying and sanitizing, drying and transferring the containers for biological samples may be repeated until the above-mentioned secondary container for the transportation of containers for biological samples is totally filled with said containers for biological samples.

Preferably, after the step of transferring said containers for biological samples into a secondary container for the transportation of containers for biological samples, a step of air-tight sealing the secondary container for the transportation of containers for biological samples is performed.

In particular, said step of air-tight sealing the above-mentioned secondary container for the transportation of containers for biological samples may be performed once it is totally filled with the containers for biological samples.

Preferably, the process for the automated sanitization of containers for biological samples provides that the above-mentioned secondary container for the transportation of containers for biological samples is previously sterilized by ultraviolet radiation.

According to a particular embodiment of the present process, the present process comprises the additional step of tracking the containers for biological samples processed along said sanitization route, preferably said tracking step being carried out by means of designated scanning means suitable for reading labels or tags present on said containers for biological samples.

According to a particularly preferred embodiment, the process according to the present invention is performed for the automated sanitization of containers for biological samples, said containers for biological samples being containers for nasal, salivary or nasal-oropharyngeal swabs, for example swabs for Covid-19 diagnosis, capillary blood samples for rapid serological test or blood sampling.

The features and advantages of the system for the automated sanitization of containers for biological samples according to the invention will be apparent from the following detailed description and from some embodiments thereof, given by way of a non-limiting example with reference to the attached drawings.

### BRIEF DESCRIPTION OF THE DRAWINGS

FIG. 1 schematically depicts a preferred embodiment of the system according to the present invention.

FIG. 2 schematically depicts a detailed perspective view of the preferred embodiment of the system according to the present invention illustrated in FIG. 1.

FIG. 3 schematically depicts a detailed sectional view of the sanitization route of the system according to the present invention illustrated in FIG. 2.

FIG. 4 schematically depicts a perspective view of an enlargement of the sanitization route and, in particular, of the packaging zone of the system according to the present invention illustrated in FIG. 2.

FIG. 5 schematically depicts a detailed perspective view of the sanitization route of the system according to the present invention illustrated in FIG. 2.

FIG. 6 schematically depicts a perspective view of an enlargement of the loading zone of the system according to the present invention illustrated in FIG. 2.

FIG. 7 schematically depicts a detailed top view of the loading zone of the enlargement relative to enlargement in FIG. 6.

FIG. 8 schematically depicts the alternative mechanism for loading containers for biological samples provided with a drawer for housing the same.

FIG. 9 schematically depicts the alternative mechanism for loading containers for biological samples provided with a housing compartment suitable for containing one or more racks for containers for biological samples.

FIG. 10 schematically depicts the alternative mechanism for loading containers for biological samples wherein the housing means comprise a drilled plate and are suitable for gripping and transferring containers for biological samples, the drilled plate performing functions of a gripper, which is not required.

### DETAILED DESCRIPTION OF PREFERRED EMBODIMENTS

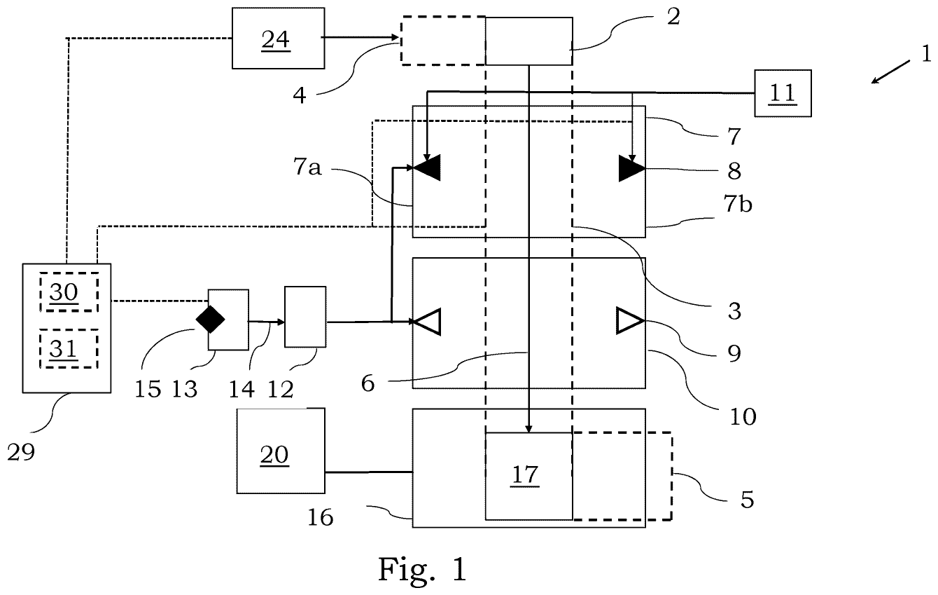

FIG. 1 illustrates a system 1 for automated sanitization of containers for biological samples according to a preferred embodiment of the present invention.

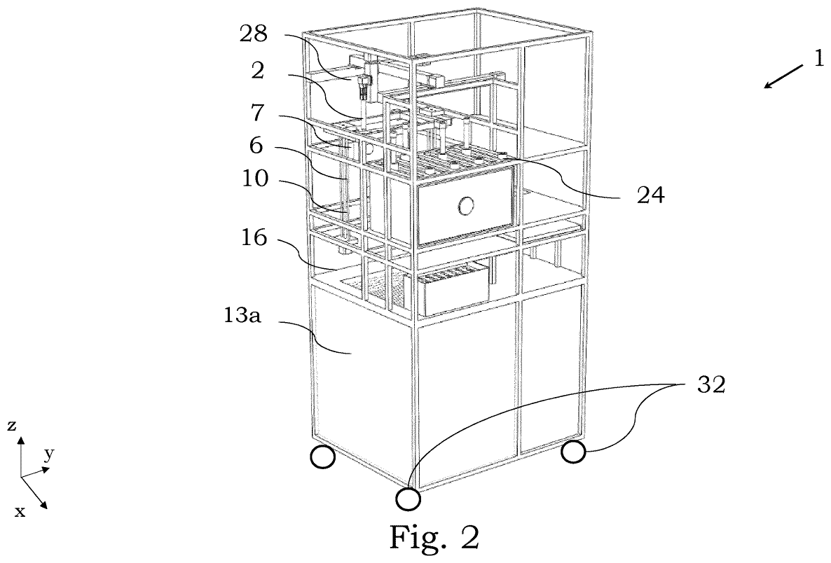

The system 1 is also illustrated in FIG. 2 in a more detailed way from the standpoint of the organization of its components in space.

The system 1 comprises, first of all, housing means 2 for containers for biological samples, a sanitization route 3 having an inlet opening 4 and an outlet opening 5, means for linearly handling 6 the housing means 2, which are suitable for conveying the containers for biological samples along said sanitization route 3, as well as a sanitization chamber 7 comprising means for dispensing 8 a biocidal fluid.

As better depicted in the next figures, the system 1 according to the present invention is suitable for sanitizing and more in general for processing containers for biological samples in the form of test tubes.

The system 1 according to the present invention also comprises means for drying 9 containers for biological samples, in the form of drying nozzles, conveniently located inside a drying chamber 10, which is also comprised in the embodiment of system 1 illustrated in FIG. 1.

Most preferably, the drying chamber 10 comprises two walls opposite each other, each of which is provided with three respective drying nozzles, to make a total of six drying nozzles.

As it is evident in FIG. 1, the sanitization chamber 7 is located upstream of the drying chamber 10 along the sanitization route 3.

Consistently, the means for dispensing 8 a biocidal fluid are located upstream of the drying means 9 along the sanitization route 3.

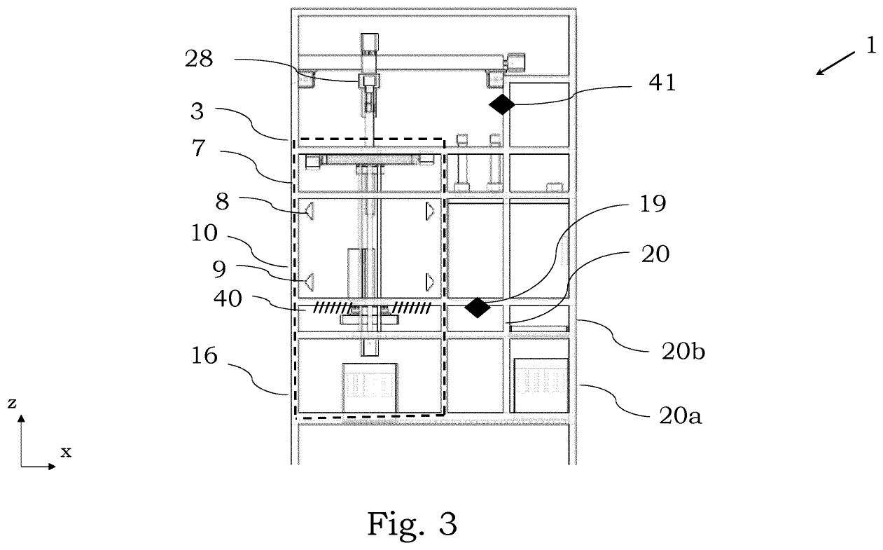

The mutual position between the means for dispensing 8 a biocidal fluid in relation to the drying means 9 along the sanitization route 3 is well depicted in FIG. 3.

With reference to the summary, the system 1 is therefore specifically structured to satisfy the need for ensuring a process for the automated sanitization of containers for biological samples.

As depicted for example in FIG. 2, the system 1 is particularly compact and easy to use.

Moreover, the overall dimension of system 1 are very small, thereby facilitating not only its use in crowded places, but also its retention and its cleaning and/or disinfection.

In particular, the system 1 according to the present invention may be easily brought to the place where it is needed, specifically to a site for collecting biological samples, by using suitable wheels 32.

To increase the safety of the operator, the wheels 32 are provided with suitable brakes for keeping the system 1 in a fixed position.

As depicted in FIG. 3, the sanitization chamber 7, the drying chamber 10 and the packaging zone 16 are placed vertically, respectively in a downward fashion along the z axis.

In particular, the sanitization route 3 starts from the sanitization chamber 7 located above, extends into the underlying drying chamber 10 and ends in the packaging zone 16, which is located exactly under the drying chamber 10.

Even if it is not evident in the figures, the means for dispensing a biocidal fluid of the system 1 are two atomizer nozzles, comprising a dispensing hole for a jet of biocidal fluid, which is not shown, having a shape suitable for providing the dispensation of a blade-shaped jet of biocidal fluid.

The dispensing hole for a jet of biocidal fluid is further suitable for dispensing a jet of biocidal fluid having an average size of fluid particles of 30 microns.

The atomizer nozzles 8 of the system 1 shown in FIG. 1 and in FIG. 3 are located on opposite walls 7a and 7b of the sanitization chamber 7.

Even if it is not evident in the figures, the atomizer nozzles 8 conveniently allow to dispense jets of biocidal fluid having a jet opening angle of 80°.

The system 1 according to the present invention further comprises a reservoir 11 suitable for containing said biocidal fluid.

In particular, the reservoir 11 has a capacity of 20 liters and allows dispensation of a biocidal fluid for about 4 hours.

Even if it is not evident in FIG. 1, the drying means 9 are suitable for dispensing a flow of compressed air at room temperature.

Consistently, the system 1 comprises a reservoir 12 for compressed air, as well as a compressor 13, which is able, by a suitable feeding pipe 14, to fill the reservoir 12 with compressed air.

Specifically, the atomizer nozzles 8 are fed with biocidal fluid and with compressed air and inside them the biocidal fluid, in this instance an aqueous solution of sodium hypochlorite having a concentration of 0.5% by weight, and compressed air are mixed.

The compressed air is supplied by feeding ducts, not shown, comprised in the system 1 and connected to the compressor 13, which is placed in a compartment 13a underlying the packaging zone 16 (see FIG. 2).

The compressor may be fed, for example, by an electrical outlet.

The compressor 13 is thus able to produce compressed air, which is useful for both the atomizer nozzles 8 and the drying nozzles 9.

Advantageously, the provision of the reservoir 12 of compressed air allows to prevent that the compressor operates during every operation in which compressed air is required, thereby reducing the total noise produced by the system 1. Specifically, the system 1 has a reservoir 12 of compressed air having a capacity of 25 liters, which conveniently allows to perform a sufficient number of completed sanitization cycles necessary for completely filling a secondary container for the transportation of containers for biological samples, said secondary container having 40 wells, wherein the housing means 2 allow the sanitization of eight containers for biological samples per cycle.

Moreover, the compressor 13 comprises a HEPA filter for filtering the air taken from the environment, which is able to ensure a passage of about 0.5% of particles of size of 0.1 micron per liter of air, also under conditions of high risk of exposure and high concentration of contaminant agents, for example viruses and/or bacteria, in the environment.

The system 1 thus comprises a packaging zone 16.

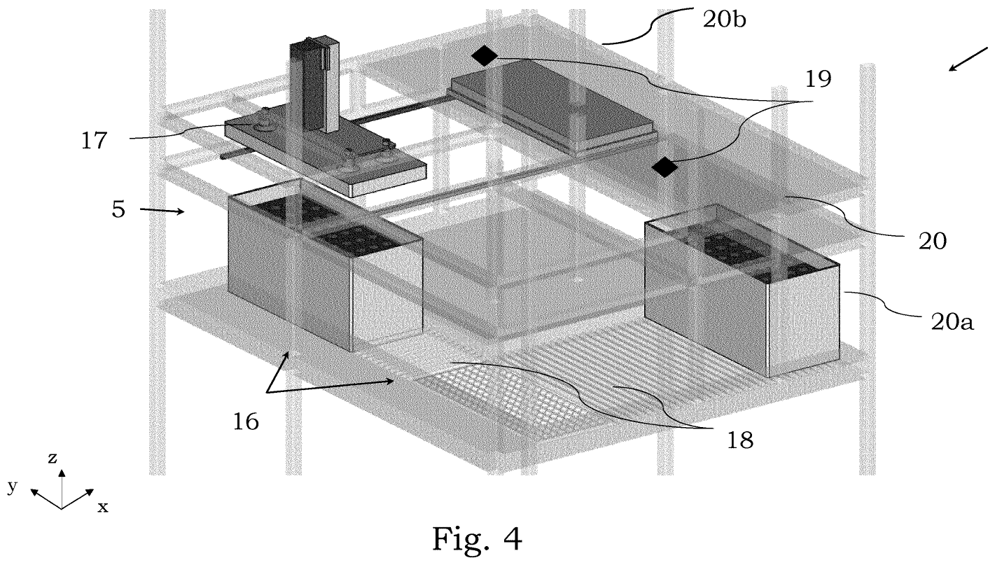

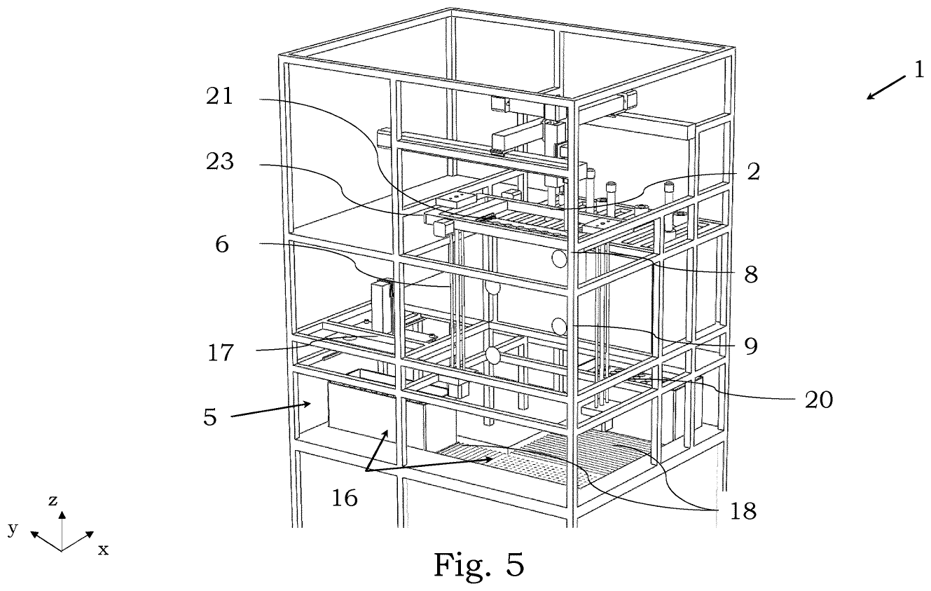

As illustrated in FIGS. 4 and 5, the packaging zone 16 comprises in turn means for packaging 17 said secondary containers.

As illustrated in FIG. 4, the means for packaging 17 are in the form of a vacuum gripper with suction cups.

Inside the packaging zone the containers for biological samples are released by the housing means 2 and placed in a secondary container for the transportation of containers for biological samples.

Specifically, the means for packaging 17 are suitable for sealing the secondary container for the transportation of containers for biological samples.

As shown in FIG. 4, the secondary container for the transportation of containers for biological samples comprises a support base and a lid.

In particular, once the secondary container for the transportation of containers for biological samples is completely filled with the containers for biological samples, in this instance after five sanitization cycles to make a total of forty containers for biological samples, the secondary container for the transportation of containers for biological samples is air-tight sealed by the means for packaging 17 by applying the lid onto the base of the secondary container.

The system 1 further comprises means for transportation 18 for a secondary container for the transportation of containers for biological samples, which are suitable to automatically convey the secondary containers from the zone of packaging 16 towards the outlet opening 5 of the sanitization route 3, so as to allow to take the containers for biological samples so-sanitized and packaged inside the secondary container.

As illustrated in FIGS. 4 and 5, the transportation means 18 for a secondary container for the transportation of containers for biological samples are also suitable for automatically conveying the base of the secondary container from a chamber of sterilization of the secondary container for the transportation of containers for biological samples towards the packaging zone 16.

In fact, the system 1 comprises a chamber for sterilization 20 of the secondary container for the transportation of containers for biological samples, wherein the sterilization chamber 20 comprises sterilization means 19 for a secondary container for the transportation of containers for biological samples which are suitable for sterilizing the surface of the secondary container.

As illustrated in FIG. 4, the sterilization means 19 are ultraviolet light lamps.

The sterilization chamber 20 for a secondary container for the transportation of containers for biological samples comprises at least one inlet opening.

In particular, the sterilization chamber 20 comprises a first inlet opening 20a, suitable to allow the operator to insert the support base of the secondary container for the transportation of containers for biological samples into the sterilization chamber 20, and a second inlet opening 20b, suitable to allow the operator to insert the lid of the secondary container into the sterilization chamber 20.

In particular, the transportation means 18 for a secondary container for the transportation of containers for biological samples, in particular means for linearly handling, suitable for automatically conveying the secondary container for the transportation of containers for biological samples from the packaging zone 16 towards the outlet opening 5 of the sanitization route 3 and the base of the secondary container from the sterilization zone 20 towards the packaging zone 16.

As shown in FIG. 4, the packaging means 17 in the form of a vacuum gripper with suction cups are also suitable for automatically conveying the lid of the secondary container for the transportation of containers for biological samples from the sterilization chamber 20 towards the packaging zone 16.

As schematically illustrated in FIG. 3, the system 1 may comprise a separation element 40, located downstream of the sanitization chamber 7 and upstream of the packaging zone 16 along the sanitization route 3.

As schematically illustrated in FIG. 3, the system 1 may comprise scanning means 41, in particular optical scanners.

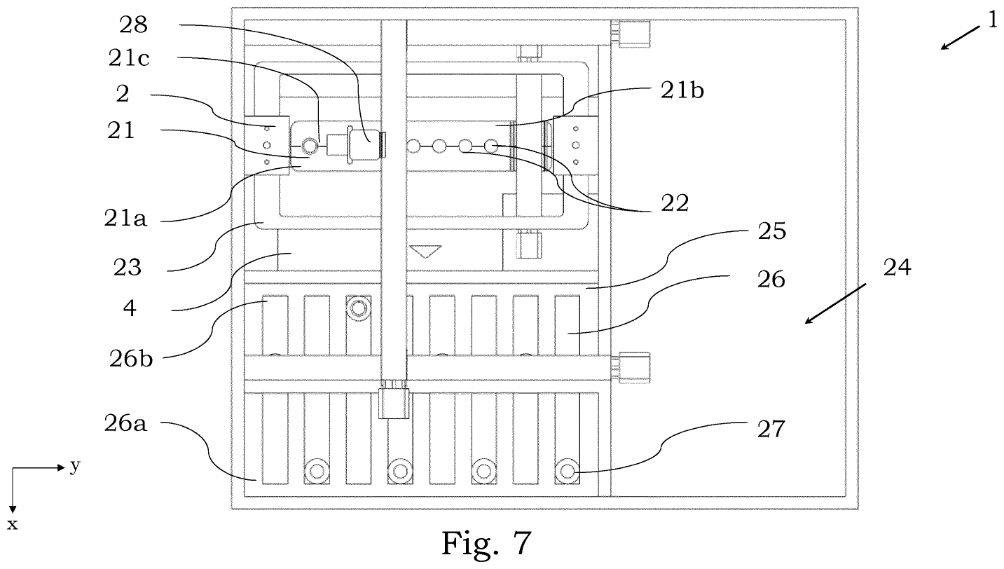

Moreover, as shown in FIGS. 5 to 7, in particular in FIG. 7, the housing means 2 for containers for biological samples comprise a drilled plate 21 comprising through holes 22 suitable for receiving the containers for biological samples.

It is evident that each through hole 22 of the drilled plate 21 is conveniently dimensioned to house a single container for biological samples.

In the present embodiment, the through holes 22 are suited to receiving containers for biological samples suitable for storage and transportation of biological samples, such as nasal, salivary or nasal-oropharyngeal swabs, capillary blood samples for rapid serological test, specifically test tubes.

The through holes 22 are suitable for accommodating containers for biological samples in an essentially vertical position.

Moreover, even if it is not illustrated, the rims of the through holes 22 are beveled and coated with gum in the zone of interface with the test tubes wall; in this way it is possible to increase the friction between the rim of the through holes 22 and the wall of the test tube, thereby minimizing the contact zone between these elements, which is inevitably more difficult to sanitize.

More specifically, the drilled plate 21 has an almost rectangular shape.

As it is evident in FIG. 7, the through holes 22 are longitudinally aligned along the length of the drilled plate 21 i.e. along the horizontal symmetry axis, which has the same direction as the y axis, shown in the figure.

The drilled plate 21 is made of metal, such as aluminum, or plastic material, more preferably of PMMA or ABS/PLA.

As shown in FIG. 7, the drilled plate 21 comprises two portions 21a and 21b which can be separated from each other along a separation line 21c defined by its horizontal symmetry axis.

As illustrated in FIGS. 5 to 7, the housing means 2 for containers for biological samples comprise a supporting frame 23, suitable for supporting the two portions which can be separated 21a and 21b of the drilled plate 21 and inside which the two portions which can be separated 21a and 21b can slide along an horizontal direction i.e. along the x axis defined in FIG. 7.

The two portions which can be separated 21a and 21b can slide independently and, according to this particular embodiment, their sliding movement is enabled by a mechanism of linear handling, such as for example recirculating ball screws or linear guides, not shown.

The housing means 2, with particular reference to the frame 23, may be let slide along the sanitization route 3, in particular from the inlet opening 4 and from the sanitization chamber 7 to the packaging zone 16, passing through the drying chamber 10, i.e. by a vertical movement along the z axis defined by the figures, thanks to the suitable means of linear handling 6.

Specifically, in the present embodiment, the means for linear handling 6 can be a linear guide, a recirculating ball bearing guide, V-belt or again a handling system provided with belts.

The guide allows to let the means for linear handling 6 slide along the sanitization route 3 according to a step-by-step movement, therefore to halt in the sanitization chamber 7 and, subsequently, in the drying chamber 10, according to the preset times.

The system of the present invention shown comprises a loading zone 24 located close to the inlet opening 4 of the sanitization route 3; the loading zone 24 is suitable for allowing to temporarily house the containers for biological samples before properly placing them in the housing means 2.

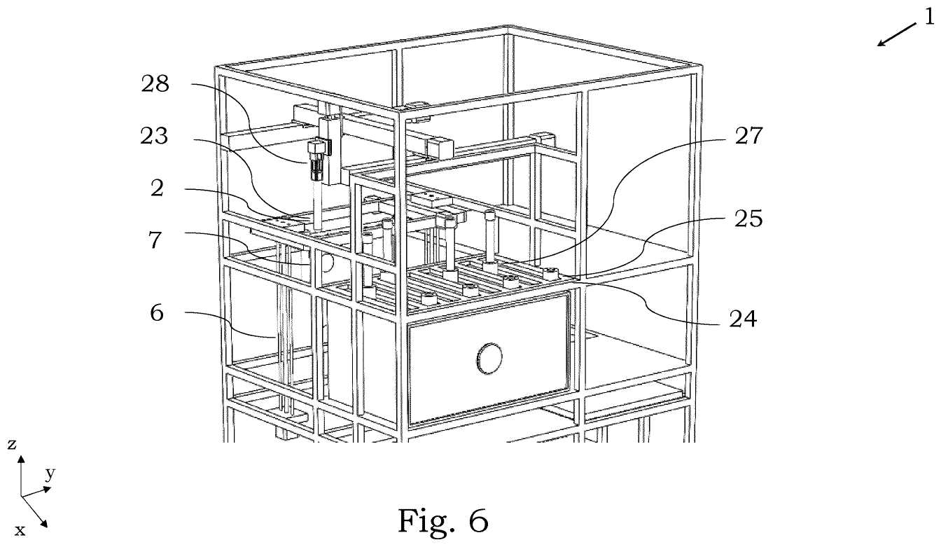

As illustrated in FIGS. 6 and 7, the loading zone 24 comprises loading means 25 for temporarily housing containers for biological samples, wherein loading means 25 are a loading grid comprising slits 26, which have a rest end 26a and an end-stroke end 26b, and loading elements 27, suitable for temporarily housing containers for biological samples, wherein each loading element 27 is conveniently dimensioned to house a single container for biological samples and can slide inside the slits 26 from the rest end 26a to the end-stroke end 26b.

As it is evident in FIG. 6, the loading elements 27 are suitable for holding the containers for biological samples in a vertical position.

Moreover, the loading means 25 comprise conveyor belts or systems for linear handling, not shown, suitable for handling said loading elements from the rest end 26a to the end-stroke end 26b and/or from the latter to the rest end 26a.

In a totally general manner, the end-stroke end 26b is conveniently located close to the inlet opening 4 of the sanitization route 3.

As it is evident in FIG. 7, the rest end 26a is in a position opposite to the end-stroke end 26b with respect to the inlet opening 4.

The embodiment of the system 1 according to the present invention shown in the figures also comprises means for transferring 28 containers for biological samples, able to pick up the latter from the loading elements 27, which have reached the end-stroke end 26b, and placing them inside the housing means 2 for containers for biological samples i.e. inside the through holes 22 of the drilled plate 21.

Specifically, the means for transferring 28 containers for biological samples are a gripper provided with flexible gripping fingers.

The system 1 according to the present invention comprises a plurality of electrical motors and specific actuators, not shown, designated for the activation of all the above-mentioned components of the system 1.

Considering again FIG. 1, the system 1 comprises also a central control unit 29 in turn comprising at least one control card 30 able to generate a command of activation for the electrical motor, the actuators and all the above-mentioned components of the system 1.

The dashed lines originating from the central control unit 29 schematically show some of the wiring present in the system 1 according to the depicted embodiment, thanks to which the so-generated command of activation can reach the above-mentioned components of the system 1 and the appropriate actuators for their activation (not shown).

The central control unit 29 also comprises a data storage unit 31 suitable for storing an operative program of functioning of the system 1.

Finally, the system 1 comprises a terminal, not shown, comprising an interface for the interaction with an operator.

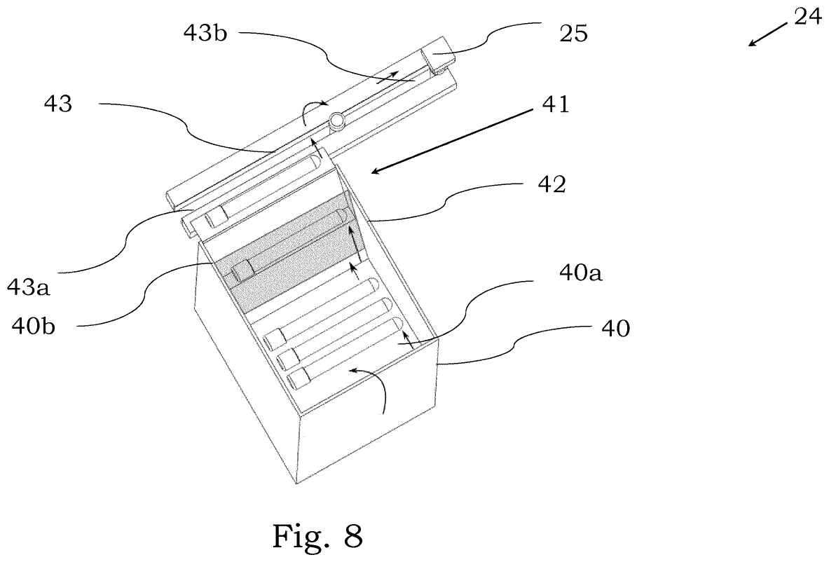

FIG. 8 represents an embodiment of the system 1 according to the present invention comprising a loading zone 24 suitable for processing randomly-organized containers for biological samples comprising a storing compartment 40 accessible to the operator, for example in the form of a drawer.

The loading zone 24 also comprises means for picking-up 41, suitable for picking-up a container for biological samples at a time from the storing compartment 40 and for conveying it towards the inlet opening 4 of the sanitization route.

The means for picking-up 41 comprises a lifting mechanism 42, suitable for taking the containers for biological samples one by one from the storing compartment 40.

The storing compartment 40 comprises a base floor 40a provided with an appropriate slope and a bottom wall 40b, perpendicular to said base floor, wherein the lifting mechanism 42 is positioned close to the zone of junction between the base floor 40a and the bottom wall 40b.

The loading zone further comprises a ramp 43, suitable for receiving one by one the containers for biological samples taken by the lifting mechanism 42, wherein the ramp 43 comprises a first end 43a in communication with the lifting mechanism 42 and a second end 43b, placed close to the inlet opening 4 of the sanitization route 3.

The ramp may alternatively be a system formed by two actuated belts located on the walls of the two section bars showed in the figure, which, being in contact with the walls of the test tube, allow its handling in a frontal direction.

In FIG. 8 are schematically illustrated also the means for transferring 28 containers for biological samples, suitable for transferring the containers for biological samples from the loading zone 24, in particular from the second end 43b, to the housing means 2.

The arrows indicate the route of loading the containers for biological samples.

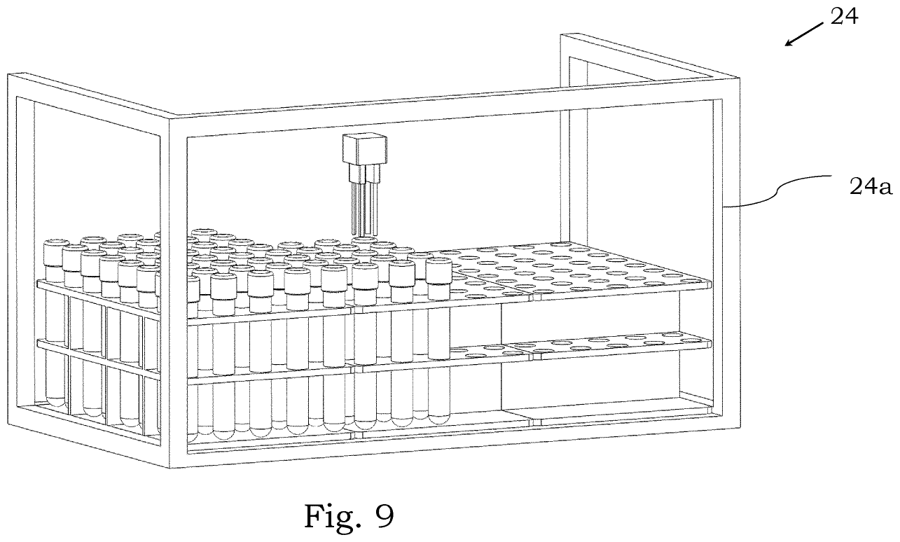

FIG. 9 shows the loading zone 24 of an embodiment of the system 1 according to the present invention comprising a housing compartment 24a suitable for containing one or more racks, for example three, for containers for biological samples.

In particular, the rack for containers for biological samples allows to hold the latter in a vertical position and is conveniently filled before being placed into the housing compartment 24a by the operator.

Clearly, according to this embodiment, the means for transferring containers for biological samples, which are suitable for transferring the containers for biological samples from the loading zone 24, are suitable for transferring the latter from the above-mentioned rack for containers for biological samples to the housing means 2.

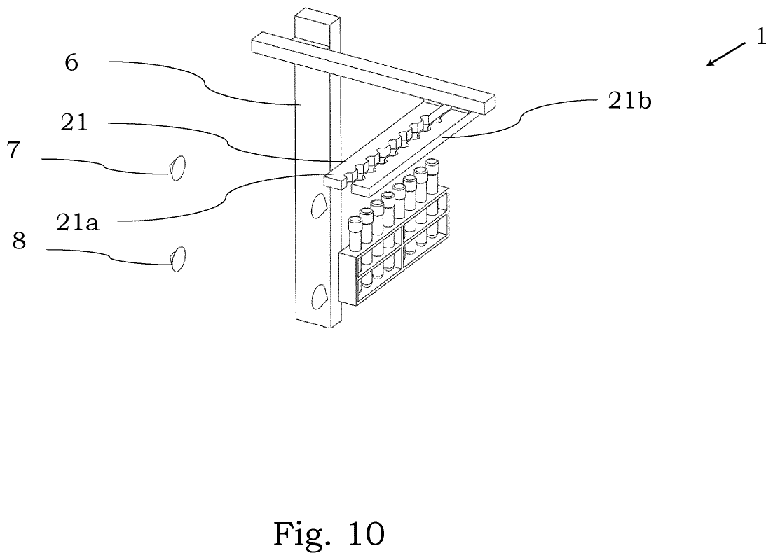

FIG. 10 shows a simplified embodiment of system 1 according to the present invention, wherein the above-mentioned housing means 2 comprise a drilled plate 21 and are suitable for gripping and transferring the containers for biological samples.

The drilled plate 21 is therefore able to pick up and transfer the containers for biological samples, for example by picking up and transferring simultaneously eight test tubes, as shown, so as to transport them through the inlet opening 4 of the sanitization route and along the same.

Specifically, bringing closer and moving apart the above-mentioned two portions which can be separated 21a and 21b of the drilled plate 21 allows in this case also to pick up the containers for biological samples.

1. A system for the automated sanitization of containers for biological samples comprising housing means for containers for biological samples, a sanitization route having an inlet opening and an outlet opening, handling means of said housing means, which are suitable for conveying the containers for biological samples along said sanitization route, a sanitization chamber and means for drying containers for biological samples, said sanitization chamber comprising means for dispensing a biocidal fluid.

2. The system according to claim 1, wherein said means for dispensing a biocidal fluid are placed upstream of said means for drying containers for biological samples along said sanitization route, said system comprising a drying chamber wherein said means for drying containers for biological samples are placed.

3. The system according to claim 1, wherein said means for dispensing a biocidal fluid are a plurality of atomizer nozzles.

4-5. (canceled)

6. The system according to claim 3, wherein said atomizer nozzles allow jets of biocidal fluid to be dispensed, said jets having a jet opening angle between 60° and 90°.

7. (canceled)

8. The system according to claim 1, comprising a packaging zone comprising means for packaging containers for biological samples, said means for packaging being suitable for packaging containers for biological samples in a secondary container for the transportation of containers for biological samples.

9. The system according to claim 8, comprising a sterilization chamber for a secondary container for the transportation of containers for biological samples, said sterilization chamber comprising sterilization means for a secondary container for the transportation of containers for biological samples suitable for sterilizing the surface of said secondary container for the transportation of containers for biological samples.

10. The system according to claim 1, wherein said housing means for containers for biological samples comprise a drilled plate comprising through holes which are suitable for receiving containers for biological samples, said through holes being dimensioned to receive a single container for biological samples.

11. The system according to claim 10, wherein said drilled plate has an oblong shape, and said through holes are longitudinally aligned along the length of said drilled plate,
said drilled plate comprising two portions which can be separated from each other along a separation line defined by the horizontal symmetry axis of said oblong shape.

12. (canceled)

13. The system according to claim 1, wherein said system comprises a loading zone placed close to said inlet opening of said sanitization route, said loading zone being suitable for allowing to temporarily house containers for biological samples before placing them in said housing means.

14. The system according to claim 13, wherein said system comprises means for transferring containers for biological samples, which are suitable for transferring containers for biological samples from said loading zone to said housing means.

15. The system according to claim 1, wherein said system comprises scanning means suitable for reading labels or tags present on said containers for biological samples.

16. A process for the automated sanitization of containers for biological samples carried out using the system according to claim 1 and comprising the following steps:
loading containers for biological samples into housing means;
conveying said so-loaded housing means along a sanitization route using handling means of the housing means and, using means for dispensing a biocidal fluid which are placed inside a sanitization chamber, applying a biocidal fluid onto said containers for biological samples;
drying said containers for biological samples, thus providing containers for biological samples thereby sanitized and dried;
optionally, transferring said containers for biological samples thereby sanitized and dried into a secondary container for the transportation of containers for biological samples.

17. The process according to claim 16, wherein said biocidal fluid is an aqueous solution of sodium hypochlorite.

18. The process according to claim 16, wherein, in said step of applying a biocidal fluid, a jet of biocidal fluid is applied and said means for dispensing a biocidal fluid are a plurality of atomizer nozzles.

19. (canceled)

20. The process according to claim 18, wherein, in said step of applying a jet of biocidal fluid, the containers for biological samples are conveyed along said sanitization route according to a step-by-step movement of said handling means and in such a way that the containers for biological samples are entirely hit by the jet of biocidal fluid, gradually all over their height.

21. The process according to claim 16, wherein, after said step of applying a biocidal fluid and before said drying step, the biocidal fluid is let act for a time period between 30 seconds and 2 minutes.

22. (canceled)

23. The process according to claim 16, wherein said process comprises an additional step of tracking said containers for biological samples processed along said sanitization route, said tracking step being carried out by means designated scanning means suitable for reading labels or tags present on said containers for biological samples.

24. The system according to claim 8, wherein said means for packaging are suitable for air-tight sealing said secondary container for the transportation of containers for biological samples.

25. The system according to claim 8, wherein said system comprises a separation element, placed downstream of said sanitization chamber and upstream of said packaging zone along said sanitization route, said separation element acting as a barrier for said biocidal fluid.

26. The system according to claim 9, wherein said sterilization means for a secondary container for the transportation of containers for biological samples are ultraviolet light lamps.
